(19) United States
(12) Patent Application Publication
(10) Pub. No.: US 2022/0346901 A1
(43) Pub. Date: Nov. 3, 2022

(54) APPARATUS FOR HOLDING MEDICAL DEVICE

(71) Applicants: MICRO-TECH (NANJING) CO., LTD., Nanjing (CN); Micro-Tech Endoscopy USA, Inc, Ann Arbor, MI (US)

(72) Inventors: Scott HAACK, Chardon, OH (US); Changqing LI, Nanjing (CN); Weiqin QIU, Nanjing (CN); Zhenghua SHEN, Nanjing (CN); Gang ZHANG, Nanjing (CN)

(21) Appl. No.: 17/348,967

(22) Filed: Jun. 16, 2021

(30) Foreign Application Priority Data
Apr. 30, 2021 (CN) 202120930490.0

(51) Int. Cl. A61B 46/10; A61B 46/00

(52) U.S. Cl. CPC A61B 46/10; A61B 46/40

(57) ABSTRACT

An apparatus for holding a medical device includes: a device sheet perpendicular to the ground; and at least one pocket layer, including a first pocket layer, where the first pocket layer is configured as a single first pocket room in its entirety and has a length enabling the medical device to be laid in the first pocket room without coiling in the presence or absence of one or more suspension elements, and the at least one pocket layer has a structure in which an edge of the at least one pocket layer facing the one or more suspension elements is open and other edges thereof are peripherally attached to the device sheet.

### CROSS-REFERENCE TO RELATED APPLICATIONS

This patent application claims priority to Chinese Patent Application No. 202120930490.0, filed on Apr. 30, 2021, the entire contents of which is hereby incorporated by reference.

### TECHNICAL FIELD

The present disclosure relates to the field of medical apparatus, and in particular, to an apparatus for holding a medical device.

### BACKGROUND

A surgery, such as minimally invasive endoscopic surgery, e.g., endoscopic submucosal dissection (ESD), endoscopic mucosal resection (EMR), endoscopic retrograde cholangiopancreatography (ERCP), is generally performed with an assistance or guidance of a medical device. The medical device may include a special catheter, a guide wire and other precision device and is generally introduced into the human body to diagnose and treat a disease in the body under a guidance of medical imaging equipment. The minimally invasive endoscopic surgery mainly uses a digital technology to expand the doctor's field of view, and it generally has characteristics of small trauma, and quick recovery.

### SUMMARY

The present disclosure provides an apparatus for holding a medical device, including:

a device sheet perpendicular to the ground; and

at least one pocket layer, including a first pocket layer,

where the first pocket layer is configured as a single first pocket room in its entirety, and has a length enabling the medical device to be laid in the first pocket room without coiling in the presence or absence of one or more suspension elements, and the at least one pocket layer has a structure in which an edge of the at least one pocket layer facing the one or more suspension elements is open and other edges thereof are peripherally attached to the device sheet.

In an embodiment, the at least one pocket layer further includes a second pocket layer disposed on a side of the first pocket layer away from the device sheet, and the second pocket layer is divided into two or more second pocket rooms. Preferably, the second pocket layer is divided into two second pocket rooms, and the two second pocket rooms may have the same or different size and shape.

In an embodiment, the at least one pocket layer further includes a third pocket layer disposed on a side of the second pocket layer away from the device sheet, the third pocket layer is divided into two or more third pocket rooms, and the two or more third pocket rooms may have the same or different size.

In an embodiment, the one or more suspension elements are used for aiding in holding the medical device, where the one or more suspension elements are disposed on the device sheet and are located above the at least one pocket layer.

In an embodiment, the one or more suspension elements are disposed close to two edges of the device sheet in a length direction of the device sheet.

In an embodiment, the one or more suspension elements include, but are not limited to, a hook, a button, a sheathe, and any combination thereof, and when there are more suspension elements, the more suspension elements are in juxtaposition in the length direction of the device sheet and are spaced apart from each other.

In an embodiment, the hook has a shape of “V”.

In an embodiment, the hook is formed by engaging a first straight branch with a curved or straight second branch in an acute angle.

In an embodiment, the hook is formed as a base including a plurality of hooks.

In an embodiment, a height of the first pocket layer is not less than that of the second pocket layer.

In an embodiment, heights of the first pocket layer, the second pocket layer, and the third pocket layer are decreased in sequence.

In an embodiment, the at least one pocket layer is quadrilateral.

In an embodiment, the at least one pocket layer and the device sheet are made from a polymer material.

In an embodiment, the at least one pocket layer is transparent, semitransparent or non-transparent.

In an embodiment, the apparatus further includes a fastener disposed on either surface of the device sheet, and used to fix the device sheet, the number of the fastener is one or more.

In an embodiment, the fastener is a velcro strip, a double-sided tape strip, or any combination thereof.

In an embodiment, the first pocket room has a length of equal to or more than 60 cm.

In an embodiment, the device sheet is coated with a waterproof or fireproof membrane.

In an embodiment, the at least one pocket layer is coated with a waterproof or fireproof membrane.

In an embodiment, the first pocket layer is provided with a connector on the surface of the first pocket layer facing the device sheet.

According to the apparatus for holding a medical device the present disclosure, it is possible to maximize unfolding of the medical device to avoid pollution or damage to the medical device due to excessive folding or cluttered placement.

### BRIEF DESCRIPTION OF DRAWINGS

In order to illustrate the embodiments of the present disclosure or the technical solutions in the prior art clearer, the accompanying drawings used in the description of the embodiments or the prior art will be briefly introduced below. Obviously, the accompanying drawings in the following description are some embodiments of the present disclosure, and other accompanying drawings that may be obtained by those skilled in the art according to the accompanying drawings without any creative work also fall into the protection scope of the present disclosure.

FIG. 1 is a schematic structural diagram of an apparatus for holding a medical device according to an embodiment of the present disclosure;

FIG. 2 is a schematic structural diagram of an apparatus for holding a medical device according to another embodiment of the present disclosure;

FIG. 3 is a schematic structural diagram of an apparatus for holding a medical device in use according to an embodiment of the present disclosure;

FIGS. 4A, 4B and 4C are schematic structural diagrams of a suspension element of an apparatus for holding a medical device according to an embodiment of the present disclosure; and

FIG. 5 is a schematic structural diagram of a fastener for fixing an apparatus for holding a medical device according to an embodiment of the present disclosure.

FIG. 6 is a schematic structural diagram of a connector on the surface of the first pocket layer facing the device sheet according to an embodiment of the present disclosure.

### DESCRIPTION OF EMBODIMENTS

In order to make the objects, technical solutions and advantages of the present disclosure clearer, the technical solutions of the embodiments of the present disclosure will be clearly and completely described below with reference to the accompanying drawings in the embodiments of the present disclosure. Apparently, the described embodiments are part of the embodiments of the present disclosure, rather than all of the embodiments. On the basis of the embodiments of the present disclosure, all other embodiments obtained by those skilled in the art without creative work fall within the protection scope of the present disclosure.

In the present disclosure, “at least one” means one or more. “And/or” describes an association relationship of the associated objects, and indicates that there may be three relationships. For example, A and/or B may indicate that A exists separately, A and B exist at the same time, and B exists separately. When an A element is referred in singular, plural reference of the A element may be included. The character “/” generally indicates that the contextual objects are in an “or” relationship. “At least one of the following” or a similar expression thereof refers to any combination of these items included, including any combination of a single item or a plurality of items. For example, at least one of a, b, or c may represents: a; b; c; a and b; a and c; b and c; or a, b and c. In the present disclosure, the terms “first”, “second”, and the like, are used to distinguish similar objects, and are not used to describe a particular order or a sequence. As used herein, the “length” refers to a distance from one side of the device sheet to the other side thereof in a direction parallel to the ground, and the “without coiling” means that the medical device may be placed without bending or with a bending that will not form a ring.

In medical treatment, specifically in surgeries, such as ESD, EMR, ERCP, and each type of surgery requires a variety of devices, for example, ESD needs to use a single use electro surgical knife, an injection needles, a disposable biopsy forcep, a clip, a polypectomy snare, and the like. For the devices, on the market, most of them have handles for hanging. However, at present, there is no suitable storage place or container for the devices in an operating room, for example endoscopic operating room, and the devices are cluttered in the operating room, a little carelessness may easily lead to cross-infection. The problem not only increase the operation time but also bring unknown potential infection risk to a patient.

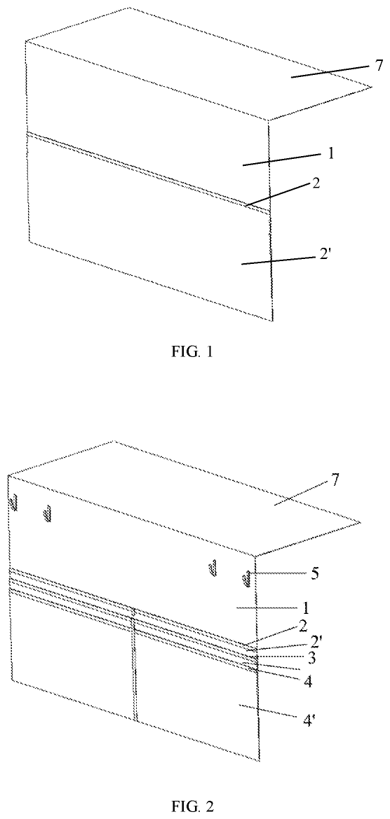

At least one embodiment of the present disclosure provides an apparatus for holding a medical device. FIG. 1 is a schematic structural diagram of an apparatus for holding a medical device according to an embodiment of the present disclosure. Exemplarily, the embodiment of FIG. 1 only shows a case of including only one pocket layer. As illustrated in FIG. 1, the apparatus includes a device sheet 1 and a first pocket layer 2 directly disposed on a side of the device sheet 1, the device sheet 1 is perpendicular to the ground and is fixed to a table 7, the first pocket layer 2 is configured as a single first pocket room 2′ and has a length enabling the medical device to be laid in the first pocket room 2′ without coiling in the presence or absence of one or more suspension elements (not shown), thereby preventing deformation, inopportune unfolding or touching the floor, and damage to the medical device. The length of the first pocket layer 2 is the same as a length of the device sheet 1, and is quadrangular. It will be appropriated that the present disclosure has no specific limitation on the shape of the first pocket layer 2, as long as the first pocket layer 2 has a structure in which an upper edge thereof is open and other edges thereof are peripherally attached to the device sheet 1. For example, the shape of the first pocket layer 2 may be an inverted triangle or in any other appropriate shape.

In an embodiment, at least one pocket layer is disposed on a side of the device sheet 1 in a direction away from the device sheet 1.

In an embodiment, at least one pocket layer, for example the first pocket layer directly disposed on the device sheet 1, an outermost pocket layer not directly disposed on the device sheet 1, or an pocket layer between the first pocket layer and the outermost pocket layer, is configured as a monolithic pocket room over an entirety length of the at least one pocket layer.

In an embodiment, the at least one pocket layer has the same length as the device sheet.

FIG. 2 is a schematic structural diagram of an apparatus for holding a medical device according to an embodiment of the present disclosure. As shown in FIG. 2, the apparatus may include a plurality of pocket layers, for example, a first pocket layer 2, a second pocket layer 3, and a third pocket layer 4. In the present embodiment, the apparatus may include one or more suspension elements 5. All details of the first pocket layer 2 as described with reference to FIG. 1 also applies to the first pocket layer 2 as shown in FIG. 2.

In an embodiment, the second pocket layer 3 is disposed on a side of the first pocket layer 2 away from the device sheet 1, and the second pocket layer 3 is divided into two or more second pocket rooms 3′ and the two or more second pocket rooms 3′ may the same or different in size and/or shape. As shown in FIG. 2, the second pocket layer 3 is disposed on the side of the first pocket layer 2 away from the device sheet 1, and the second pocket layer 3 is divided into two second pocket rooms 3′, each of second pocket rooms 3′ is used for holding one or more, preferably one medical device that has a length equal to or slightly greater than a length of each of the second pocket rooms 3′. In an embodiment, the second pocket rooms 3′ are the same or different.

In an embodiment, the third pocket layer 4 is disposed on a side of the second pocket layer 3 away from the device sheet 1, and the third pocket layer 4 is divided into two or more third pocket rooms 4′, and the two or more third pocket rooms 4′ may the same or different in size and/or shape. As shown in FIG. 2, the third pocket layer 4 is disposed on the side of the first pocket layer 2 away from the device sheet 1, and the third pocket layer 4 is divided into two third pocket rooms 4′, each of third pocket rooms 4′ is used for holding one or more, preferably one medical device that has a length equal to or slightly greater than a length of each of each of the third pocket rooms 4′.

In an embodiment, the third pocket room 4's are the same or different, and the third pocket rooms 4′ may be design as the same with the second pocket rooms 3′, or the third pocket rooms 4′ may be design as different from the second pocket rooms 3′.

During a minimally invasive endoscopic surgery, a medical device such as a single use electro surgical knife, an injection needle, a disposable biopsy forcep, a clip, a polypectomy snare is needed, the medical device may be hold in the first pocket room 2′ that is a monolithic pocket room over an entirety length of the first pocket layer 2, one of the two or more second pocket rooms 3′, or one of the two or more third pocket rooms 4′, depending on a length of the medical device.

It should be understood that the present disclosure is not limited to three pocket layers, more than three pocket layers may be sequentially disposed on a side of the device sheet 1 in a direction away from the device sheet 1 and divided into two or more pocket rooms, which may be designed according actual need, allowing more medical devices to be held.

In an embodiment, the at least one pocket layer may be a quadrilateral, an inverted triangle or any other appropriate shape.

In an embodiment, the at least one pocket layer is waterproof and fireproof.

In an embodiment, the at least one pocket layer has a length same as a length of the device sheet 1.

In an embodiment, the apparatus further includes one or more suspension elements 5 for aiding in holding the medical device and receiving part of the medical device that is longer than the length of a corresponding pocket room, where the one or more suspension elements 5 are disposed on the device sheet 1 and are located above the at least one pocket layer. Part of the medical device exposed outside a pocket room may be hung on a suspension element 5 by a handle of medical device or an appropriate auxiliary tool.

In an embodiment, the one or more suspension elements 5 are disposed close to two edges of the device sheet 1 in a length direction of the device sheet 1. For example, two suspension elements 5 are disposed close to a first edge of the device sheet 1 in a length direction of the device sheet 1, another two suspension elements 5 are disposed close to a second edge of the device sheet 1 in a length direction of the device sheet 1. In this case, the handle of the medical device is engaged with a corresponding suspension element 5, allowing main bodies or most of the medical device to be inside a corresponding pocket room, from one side of the corresponding pocket room to the other side thereof.

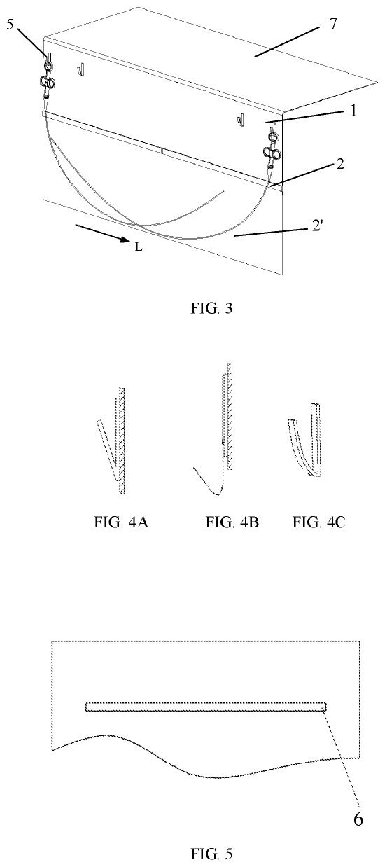

FIG. 3 is a schematic structural diagram of an apparatus for holding a medical device in use according to an embodiment of the present disclosure. As illustrated in FIG. 3, above the first pocket room 2′ (as illustrated with reference to FIG. 1) that is a monolithic pocket room, there are four suspension elements 5 disposed on the device sheet 1, two are close to the first edge of the device sheet 1 in a length direction L of the device sheet 1, the other two are close to the second edge of the device sheet 1 in a length direction L of the device sheet 1.

In an embodiment, each pocket room may receive at least one medical device, preferably one medical device. As shown in FIG. 3, the first pocket room 2′ receive two medical devices, where one medical device is engaged with one suspension element 5 close to left side of device sheet 1 by a handle of the medical device, and the other medical device is engaged with another suspension element 5 close to right side of device sheet 1 by a handle of the medical device, leaving main bodies of the two medical devices within one first pocket room 2′ from one side to the other side along a length of the one first pocket room 2′.

In a case of more suspension elements, the more suspension elements 5 are in juxtaposition in the length direction L (as illustrated in FIG. 3) of the device sheet 1 and are spaced apart from each other. For example, distances between the more suspension elements 5 may be the same or different and preferably symmetrically distributed on both sides of the device sheet 1. For another example, heights of the more suspension elements 5 from the first pocket room 2′ may be the same or different.

The one or more suspension elements 5 may be constructed into different types. In an embodiment, the one or more suspension elements 5 include a hook, a button, a sheath, or any combination thereof. For example, the one or more suspension elements 5 are hooks, the handles of the medical devices are hung on the hooks, as shown in FIG. 3.

In an embodiment, the hooks have a shape of “V”. For example, one branch of “V” is fixed on the device sheet 1, the other branch of “V” is used to engage the handle of the medical device.

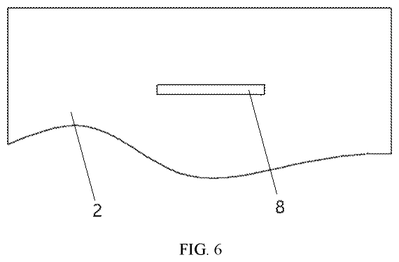

The hook in “V” shape is formed by engaging a first straight branch with a curved or straight second branch in an acute angle so that the medical device may be hung onto the hook. FIGS. 4A, 4B and 4C are schematic structural diagrams of an apparatus for holding a medical device according to an embodiment of the present disclosure. As illustrated in FIGS. 4A-4C, for example, a first straight branch is fixed on the device sheet 1, a straight second branch (biased; as can be seen in FIG. 4A) is used to engage the handle of the medical device. For another example, a first straight branch (vertical) is fixed on the device sheet 1, a curved second branch (as can be seen in FIG. 4C) is used to engage the handle of the medical device. The hook having a shape of “V” may maintain the medical device to engage the hook, not falling off the hook.

In an embodiment, the hook is formed as a base including a plurality of hooks thereon, and the hooks also have a “V” shape. For example, the plurality of hooks are each used to engage a handle of the medical device, and the base is fixed onto the device sheet 1.

In an embodiment, a height of an inner pocket layer is always equal to or larger than that of an outer pocket layer so that the outer pocket layer will not hinder the medical device from putting into a corresponding pocket room of the inner pocket layer.

For example, a height (relative to the ground) of the first pocket layer 2 is not less than that of the second pocket layer 3, so that the second pocket layer 3 will not hinder the medical device from putting into a corresponding pocket room of the first pocket layer 2.

For another example, heights of the first pocket layer 2, the second pocket layer 3, and the third pocket layer 4 are decreased in sequence, so that corresponding medical devices may be put into corresponding pocket rooms easily. For example, the first pocket layer 2 has a first height; the second pocket layer 3 has a second height which is lower than the first height, allowing a medical device putting into a corresponding second pocket room 3′ easily; similarly, the third pocket layer 4 has a third height which is lower than the second height, allowing a medical device putting into a corresponding third pocket room 4′ easily.

In an embodiment, the at least one pocket layer is quadrilateral, an inverted triangle or in any other appropriate shape. For example, the at least one pocket layer is square or rectangle.

In an embodiment, the at least one pocket layer has a structure in which an edge thereof facing the one or more suspension elements 5 is open and the other edges thereof are attached or sealed to device sheet 1, which allow the medical device may be placed into a corresponding pocket room.

In an embodiment, the at least one pocket layer and the device sheet 1 are made from a polymer material. For example, the at least one pocket layer and the device sheet 1 are made from polyethylene, polypropylene, polystyrene, polymethyl, methacrylate, polyvinyl, chloride, or any combination thereof, and both of them may be made of the same or different materials.

In an embodiment, the device sheet may have a hardness higher than the at least one pocket layer.

In an embodiment, the at least one pocket layer is transparent, semitransparent or non-transparent. If the at least one pocket layer is transparent, it is easy to observe and thereby quickly and accurately take out a desired medical device, and if the pocket is non-transparent, it is easy to hide the contents inside the pocket room to avoid being found and taken away.

In an embodiment, the device sheet 1 is coated with a waterproof or fireproof membrane. As the medical device may be used to receive a body fluid, and the body fluid may wet the device sheet 1, the waterproof membrane may prevent the device sheet 1 from getting wet and contaminated. In an embodiment, the at least one pocket layer is waterproof, which may also prevent the at least one pocket layer from getting wet and contaminated.

During the minimally invasive endoscopic surgery, the device sheet 1 is fixed on any appropriate place, for example on doctor, such as a cart, table, or wall. In an embodiment, the apparatus further includes a fastener disposed on either surface of the device sheet 1, and the fastener is used to fix the device sheet 1, the number of the fastener is one or more and there is no specific limitation as long as the device sheet 1 can be firmly fixed and will not hinder placement of the medical device. For example, the fastener may be disposed on an upper part of a surface of the device sheet 1 having the at least one pocket layer. For another example, the fastener is disposed on an upper part of a surface of the device sheet 1 without the at least one pocket layer.

In an embodiment, the fastener is a velcro strip, a double-sided tape strip, or any combination thereof. FIG. 5 is a schematic structural diagram of a fastener for fixing an apparatus for holding a medical device according to an embodiment of the present disclosure. As illustrated in FIG. 5, the fastener is a double-sided tape strip disposed on a surface of the device sheet 1 away from the at least one pocket layer.

In an embodiment, the first pocket room 2′ has a length of equal to or more than 60 cm. For example, this can make that the medical device, which is generally has a length of 60-110 cm, may be held by the first pocket room 2′ with aid of one or more suspension elements. In other embodiment, the first pocket room 2′ may have another length, for example, as long as the length of the table 7 (≥60 cm) attach to.

In an embodiment, FIG. 6 is a schematic structural diagram of a connector on the surface of the first pocket layer facing the device sheet according to an embodiment of the present disclosure. As illustrated in FIG. 6, the first pocket layer is provided with a connector 8 on the surface of the first pocket layer 2 facing the device sheet 1 for fixing the first pocket layer 2 to the device sheet 1, the connector 8 may attach the first pocket layer 2 to the device sheet 1 partially. For example, the connector 8 may be a velcro, which is disposed on the middle portion of the edge of the first pocket layer 2, the velcro may attach the first pocket layer 2 to the device sheet 1 after the medical device put into the first pocket room 2′, which avoid the first pocket room 2′ as well as the second pocket rooms 3′ and the third pocket rooms 4′ to incline outward, and the first pocket room 2′ as well as the second pocket rooms 3′ and the third pocket rooms 4′ can be attached to the device sheet 1 well, so that the medical device is easy to be held by the second pocket rooms 3′ and the third pocket rooms 4′. In another embodiment, the connector 8 may be other elements that can fix the first pocket layer 2 to the device sheet 1, and there is no limitation.

In some embodiments of the apparatus for holding a medical device of the present disclosure, the second pocket layer 3 with two or more second pocket rooms 3′ is disposed on a side of the first pocket layer 2 away from the device sheet 1, the third pocket layer 4 with two or more third pocket rooms 4′ is disposed on a side of the second pocket layer 3 away from the device sheet 1, such that cross-infection during surgery is effectively avoided.

It should be noted that the above embodiments are merely intended to illustrate the technical solutions of the present disclosure, but not to limit thereto; although the present disclosure is illustrated in detail with reference to the foregoing embodiments, those skilled in the art should understand that they may still make modifications to the technical solutions described in the foregoing embodiments or equivalent substitutions to part or all technical features therein; however, these modifications or substitutions do not make the essence of the corresponding technical solutions depart from the scope of the solutions of the embodiments of the present disclosure.

What is claimed is:

1. An apparatus for holding a medical device, comprising:
a device sheet perpendicular to the ground;
at least one pocket layer, comprising a first pocket layer, wherein the first pocket layer is configured as a single first pocket room in its entirety and has a length enabling the medical device to be laid in the first pocket room without coiling in the presence or absence of one or more suspension elements, and the at least one pocket layer has a structure in which an edge of the at least one pocket layer facing the one or more suspension elements is open and other edges thereof are peripherally attached to the device sheet.

2. The apparatus according to claim 1, wherein the at least one pocket layer further comprises a second pocket layer disposed on a side of the first pocket layer away from the device sheet, and the second pocket layer is divided into two or more second pocket rooms.

3. The apparatus according to claim 2, wherein the at least one pocket layer further comprises a third pocket layer disposed on a side of the second pocket layer away from the device sheet, and the third pocket layer is divided into two or more third pocket rooms.

4. The apparatus according to claim 1, wherein the at least one pocket layer has the same length as the device sheet.

5. The apparatus according to claim 1, wherein the one or more suspension elements are used for aiding in holding the medical device, wherein the one or more suspension elements are disposed on the device sheet and are located above the at least one pocket layer.

6. The apparatus according to claim 5, wherein the one or more suspension elements are disposed close to two edges of the device sheet in a length direction of the device sheet.

7. The apparatus according to claim 6, wherein the one or more suspension elements comprise a hook, a button, a sheathe, or any combination thereof, and when there are more suspension elements, the more suspension elements are in juxtaposition in the length direction of the device sheet and are spaced apart from each other.

8. The apparatus according to claim 7, wherein the hook has a shape of “V”.

9. The apparatus according to claim 8, wherein the hook is formed by engaging a first straight branch with a curved or straight second branch in an acute angle.

10. The apparatus according to claim 8, wherein the hook is formed as a base comprising a plurality of hooks.

11. The apparatus according to claim 3, wherein a height of the first pocket layer is not less than that of the second pocket layer.

12. The apparatus according to claim 3, wherein heights of the first pocket layer, the second pocket layer 3, and the third pocket layer are decreased in sequence.

13. The apparatus according to claim 1, wherein the at least one pocket layer is quadrilateral.

14. The apparatus according to claim 1, wherein the at least one pocket layer and the device sheet are made from a polymer material.

15. The apparatus according to claim 1, wherein the at least one pocket layer is transparent, semitransparent or non-transparent.

16. The apparatus according to claim 1, further comprising: a fastener disposed on either surface of the device sheet, and used to fix the device sheet, the number of the fastener is one or more.

17. The apparatus according to claim 16, wherein the fastener is a velcro strip, a double-sided tape strip, or a combination thereof.

18. The apparatus according to claim 1, wherein the first pocket room has a length of equal to or more than 60 cm.

19. The apparatus according to claim 1, wherein the device sheet is cover with a waterproof membrane.

20. The apparatus according to claim 1, wherein the at least one pocket layer is waterproof.

21. The apparatus according to claim 1, wherein the first pocket layer is provided with a connector on the surface of the first pocket layer facing the device sheet for fixing the first pocket layer to the device sheet.
